(19) United States
(12) Patent Application Publication
(10) Pub. No.: US 2010/0057153 A1
(43) Pub. Date: Mar. 4, 2010

(54) ELECTROMAGNETIC INTERFERENCE ALARM

(75) Inventor: Joseph J. Ballis, Shoreview, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/548,850

(22) Filed: Aug. 27, 2009

Related U.S. Application Data
(62) Division of application No. 10/449,428, filed on May 30, 2003, now abandoned.

(51) Int. Cl. A61N 1/08

(52) U.S. Cl. 607/5; 607/63; 607/17

(57) ABSTRACT

An apparatus and method are disclosed including an implantable medical device electrically coupled to a patient, having a sensor for sensing physiologic conditions and circuitry coupled to the sensor for emitting therapy in response to sensed physiologic conditions. A detector is coupled to the cardiac device for detecting the presence of electromagnetic interference and the intensity thereof and an alarm is coupled to the detector to signal the patient of the implantable medical device of the presence of electromagnetic interference.

### CROSS-REFERENCE TO RELATED APPLICATIONS

This application is a divisional of U.S. patent application Ser. No. 10/449,428 filed on May 30, 2003. The disclosure of the above application is incorporated herein by reference.

### FIELD OF THE INVENTION

The present invention generally relates to electromagnetic interference alarms, and more particularly relates to electromagnetic interference alarms in implantable medical devices.

### BACKGROUND OF THE INVENTION

An implantable medical device (IMD) may be a device such as an implantable pulse generator (IPG), commonly referred to as a pacemaker, which is used to stimulate the heart into a contraction if the sinus node of the heart is not properly timing, or pacing, the contractions of the heart. Modern cardiac devices also perform many other functions beyond that of pacing. For example, some cardiac devices such as implantable cardioverter-defibrillators (ICDs) may also perform therapies such as defibrillation and cardioversion as well as providing several different pacing therapies, depending upon the needs of the user or patient and the physiologic condition of the patient's heart.

Examples of other IMDs include various physiological stimulators including nerve, muscle, and deep brain stimulators, various types of physiological monitors and drug delivery systems, just to name a few. For convenience, all types of implantable medical devices will be referred to herein as IMDs, it being understood that the term, unless otherwise indicated, is inclusive of an implantable device capable of administering any of a number of therapies to the patient. Therefore, while implantable medical devices (IMDs) have many functions as noted above, for purposes of this application reference will be made only to implantable cardiac devices and particularly to implantable cardiac pacemakers or defibrillators, it being understood that the principles herein may have applicability to other implantable medical devices as well.

In typical use, a pacemaker or defibrillator device is implanted in a convenient location usually under the skin of the patient and in the vicinity of the one or more major arteries or veins. One or more electrical leads connected to the device are inserted into or on the heart of the patient, usually through a convenient vein. The ends of the leads are placed in contact with the walls or surface of one or more chambers of the heart, depending upon the particular therapies deemed appropriate for the patient.

One or more of the leads is adapted to carry a current from the pacemaker to the heart tissue to stimulate the heart in one of several ways, again depending upon the particular therapy being delivered. The leads are simultaneously used for sensing the physiologic signals provided by the heart to determine when to deliver a therapeutic pulse to the heart, and the nature of the pulse, e.g., a pacing pulse or a defibrillation shock.

The sensing of the physiologic signal from the heart requires a very sensitive sensing method since the signals sensed are of quite low amplitude. The presence of electromagnetic interference (EMI), if the field is large enough, can compromise the cardiac sensing function such that the pacemaker may fail to deliver a needed therapy or may deliver an unwanted therapy. Some forms of EMI are not easily distinguishable from physiologic signals and therefore can be confused with the desired physiologic signals. There is no way to single out this interference. Other types of EMI, such as continuous wave at high frequencies, can easily be distinguished from physiologic signals (non-physiologic EMI). However, if large enough they can block the sensing of the physiologic signals and leave the device without the needed information to reliably treat the heart. Other sources of disruptive interference can also compromise the sensing of the physiologic signals by an IMD.

Presently, in the case of many Bradycardia pacemakers (for correcting a slow heartbeat) and some tachycardia pacemakers (for correcting a rapid heartbeat), the pacemakers revert to a fixed pulse rate when EMI blocks or overrides the physiologic signals. This may not be optimal therapy. Among the complications are; the fixed rate may cause an arrhythmia in some patients; or in the case of tachycardia therapies the device may miss the need for delivery of a therapy.

Accordingly, devices and methods have been proposed for detecting non-physiologic EMI and providing a warning to a patient using such an IMD. In the past however such devices and methods have provided only a simple alarm that, although providing a warning to a patient that he has entered an area in which EMI may affect the functioning of his IMD, these devices and methods did not assist the patient in moving to an area where EMI is not problematical. Thus it would be useful to provide an EMI detection alarm for IMDs that also, by varying the nature of the alarm, assists the patient in moving from an area of EMI. Furthermore, other desirable features and characteristics of the present invention will become apparent from the subsequent detailed description of the invention and the appended claims, taken in conjunction with the accompanying drawings and this background of the invention.

### SUMMARY OF THE INVENTION

An apparatus and method are provided for detecting non-physiologic electromagnetic interference (EMI) to an implantable medical device (IMD) and for warning the user of the device of the danger of remaining in the vicinity of the source of the EMI, such that the patient could move away from the area to restore proper operation of the pacemaker. The apparatus comprises an implantable medical device electrically coupled to a patient, having a sensor for sensing physiologic conditions and circuitry coupled to the sensor for providing therapy in response to sensed physiologic conditions. A detector is coupled to the device for detecting the presence of electromagnetic interference and an alarm is coupled to the detector to signal the patient of the implantable medical device of the presence of electromagnetic interference and the intensity thereof and to assist the patient in avoiding the area of disruptive EMI.

### BRIEF DESCRIPTION OF THE DRAWINGS

The present invention will hereinafter be described in conjunction with the following drawing figures, wherein like numerals denote like elements, and

FIG. 1 is a diagram showing the typical placement of an IMD in a patient;

FIG. 2 is a block diagram of a pacemaker according to the present invention;

FIG. 3 is a block diagram of an electromagnetic interference detector and alarm circuit according to the present invention; and

FIG. 4 is flow chart describing the method of operation of the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

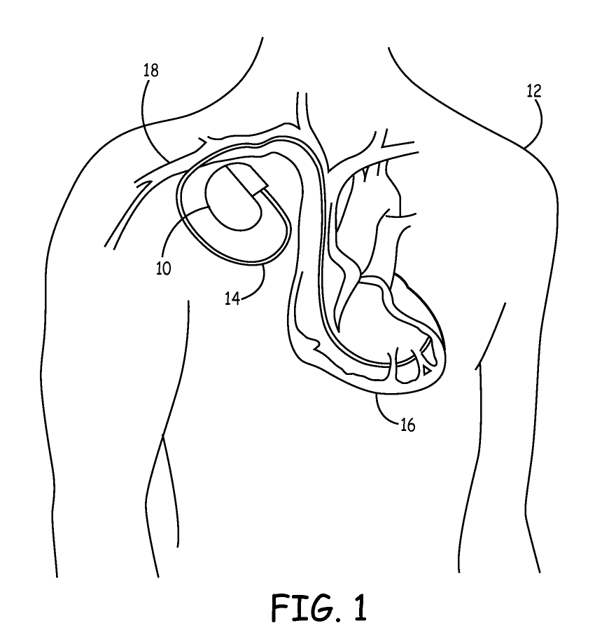

FIG. 1 is an illustration showing generally where an implantable medical device (IMD) 10 is placed in a conventional manner in a patient 12. IMD 10 is conventionally housed within a hermetically sealed, biologically inert outer canister, which itself may be of a conductive material and serve as an electrode in the IMDs pacing/sensing circuit. One or more leads, collectively identified as 14 are electrically coupled to IMD 10 in a conventional manner, extending into the patient's heart 16 via a vein 18. Disposed generally near the distal end of lead 14 are one or more exposed conductive electrodes for receiving electrical cardiac signals and/or for delivering electrical stimuli or other therapies to heart 16. Lead 14 may be implanted with its distal end in either the atrium or the ventricle of heart 16. Each of the lead 14s is preferably a bipolar lead such that each lead 14 actually has two separate and mutually insulated leads, the first having a terminal at the distal end of lead 14 and the second having a terminal near, but set back from the distal end. Such leads are well known in the art.

An implantable medical device (IMD) may have a pulse generator for producing pulses that are used to pace the heart, that is, to cause a depolarization of the heart tissue or to issue a defibrillation pulse to shock the heart from an arrhythmia to a normal heart beat. A processor within the IMD analyzes the sensed pulses to determine whether a therapy should be administered. As noted above, although the present invention may have applicability to a number of types of implantable medical devices, the following description will utilize an exemplary IMD.

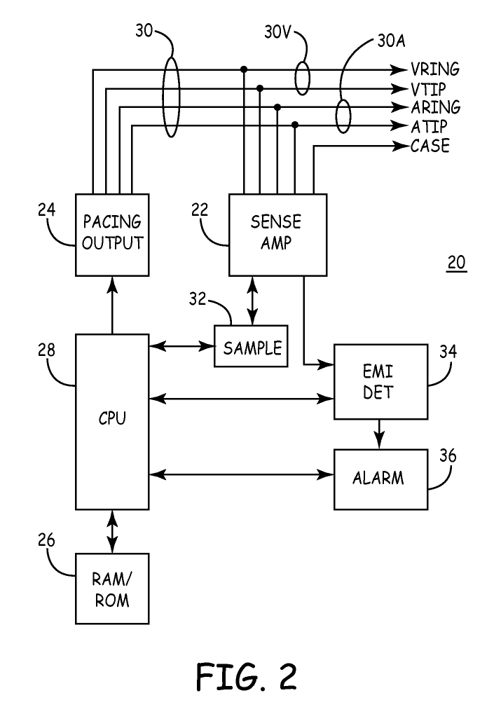

FIG. 2 is a block diagram of an implantable medical device usable in the present invention. While the device of FIG. 2 is shown as a pacemaker, it is understood that other devices could also be used, including devices such as ICD, IPG, and the like. Additionally, although the device of FIG. 2 shows an electromagnetic interference detector, it is understood that other circuits for detecting different phenomena that could affect the sensing circuit of the ICD may also be included within the invention.

The IMD comprises a primary pacing/control circuit 20. Much of the circuitry associated with pacing control circuit 20 may be of conventional design in accordance, for example, with U.S. Pat. No. 5,534,018, assigned to the assignee of the present invention, and which patent is incorporated by reference herein in its entirety, including those documents incorporated into that patent by reference. According to the present invention, the pacemaker includes an EMI detector 34 for detecting the presence of non-physiologic electromagnetic interference and activating an alarm 36 to warn the user of the pacemaker that he is in the presence of a disruptive EMI signal.

To the extent that certain components of the IMD are conventional, they will not be described in great detail here, since it is believed that the design and implementation of such components would be a matter of routine to those of ordinary skill in the art. For example, the IMD includes a sense amplifier circuit 22, pacing output circuit 24, a random access memory and read only memory (RAM/ROM) unit 26, and a central processing unit (CPU) 28.

The IMD is coupled to leads 30 which, when implanted, extend transvenously between the implant site of IMD 10 (FIG. 1) and the patient's heart. FIG. 2 shows leads 30 being coupled, either directly or indirectly to sense amplifier 22 and pacing output circuit 24, in accordance with common practice, such that cardiac electric signals may be conveyed to sensing circuitry 22 and pacing pulses from pacing output circuit 24 may be delivered to cardiac tissue, via leads 30. In the interest of clarity it should be understood that a modern IMD may contain additional circuitry not shown in FIG. 2, such as a crystal oscillator for pacing rate control, or telemetry circuitry which allows IMD 10 to be diagnosed and reprogrammed externally after implant.

In the present embodiment two bipolar leads are employed, an atrial lead 30A having atrial tip and ring electrodes (ATIP and ARING), and a ventricular lead 30V having ventricular tip and ring electrodes (VTIP and VRING). Those of ordinary skill in the art will appreciate that a separate, electrically insulated conductor extending along the length of leads 30A and 30V is associated with each of the electrodes ATIP, ARING, VTIP, and VRING. That is, electrical signals applied, for example to the VRING electrode are conducted along lead 30V on a first conductor, whereas signals applied to the VTIP electrode are conducted along a second, separate conductor in lead 30V. In addition, as noted above, the conductive, hermetically sealed canister of IMD 10 (not shown) may serve as an indifferent electrode (CASE in FIG. 2).

As previously noted, central processing unit 28 may be an off-the-shelf microprocessor or microcontroller. Although as also previously noted, specific connections between CPU 28 and the other components of the IMD may not be shown in FIG. 2, it will be apparent to those skilled in the art that CPU 28 functions to control the timed operations of pacing output circuit 24 and sense amplifier circuit 22 under control of programming algorithms stored in RAM/ROM 26. A crystal oscillator circuit (not shown) provides the main timing clock signals to the IMD. It is also understood that the circuitry of the IMD is powered by a battery inside the hermetically sealed case of the IMD in accordance with common practice in the art. For the sake of clarity, the battery and the connections between the battery and the various circuit elements are not shown.

Pacing output circuit 24, which functions to generate pacing stimuli under control of signals issued by CPU 28, may be, for example, of the type disclosed in U.S. Pat. No. 4,476,868 to Thompson, entitled “Body Stimulator Output Circuit,” which patent is hereby incorporated herein by reference in its entirety. Again, however, it is believed that those of ordinary skill in the art could select from among many various types of prior art pacing output circuits which would be suitable for the purposes of practicing the present invention.

With continued reference to FIG. 2, sense amplifier circuit 22 includes lead circuitry that essentially functions as a multiplexer to selectively couple the lead conductors associated with the ATIP, ARING, VTIP, and VRING electrodes of leads 30A and 30V to the remaining components of ICD 10 FIG. 1).

Coupled to sense amplifier 22 is an excitation and sample circuit 32 which functions to generate electrical excitation pulses which are conveyed along leads 30A and 30V for the purposes of measuring impedance between various combinations of electrodes ATIP, ARING, VTIP, and VRING, and, in addition, excitation and sample circuit 32 performs a sampling function on electrical signals present on the conductors of leads 30A and 30V.

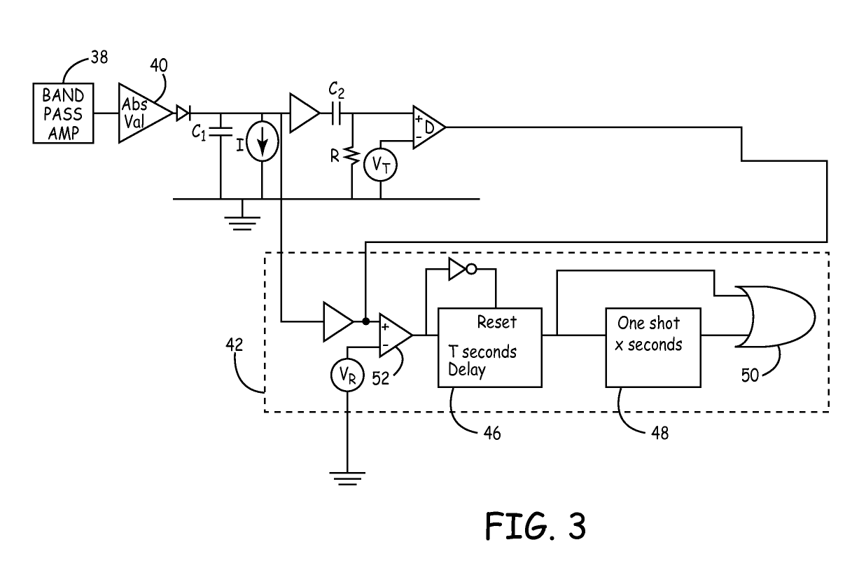

FIG. 2 also shows an EMI detector 34 coupled to sense amplifier 22 and also to an alarm circuit 36. The alarm itself can have many different forms in accordance with design choice and the desires of the user of the pacemaker device. For example, the alarm may be in the form of a low frequency, low-level tactile (i.e., vibratory) stimulation at the implant site, that is, the site where the ICD is implanted in the patient's body, or elsewhere on the patient's body. Alternatively, the alarm could be in the form of an audible alarm. Some locations where the presence of disruptive EMI may exist may also be noisy environments (shops, markets, parking lots, train yards, sidewalks, etc.) so the use of a tactile alarm may be preferred. It is important only that the patient be made aware of the presence of the disruptive EMI so the patient can move until the deleterious EMI abates. The alarm, whether stimulative or auditory or otherwise (for example, a visual signal displayed on an external device telemetrically linked to the IMD) is designed and coupled in such a manner as to provide an indication to the patient that he is in an area of EMI, and also to provide an indication to the patient whether he is being increasingly or decreasingly subjected to EMI so that he may take appropriate actions to leave the EMI area, or otherwise eliminate the source of the EMI (e.g., turn off an appliance). The operation of the alarm will be discussed more fully with respect to FIG. 3.

FIG. 3 is a circuit diagram of an EMI detector and alarm circuit usable in the instant invention. The physiologic signals from sense amplifier 22 of FIG. 2, which includes R wave signals that must be detected in order to determine whether to provide a pacing pulse or not, is provided to bandpass amplifier 38. The modified physiologic signal is then output to an absolute value circuit 40 to obtain the absolute value of the physiologic signals.

The values of capacitor C1 and current source I are chosen such that the voltage drop between outputs is less than a threshold voltage (V_{T}) for output frequencies above a predetermined value (f_{cutoff}). This eliminates the sense of signals above the predetermined value (f_{cutoff}). An advantage of the circuit of FIG. 3 is that the R-wave can be sensed in the presence of low-level EMI. The voltage on capacitor (C1) reaches a steady state value in the presence of EMI and the value of the R-wave would add on top of the EMI until the range of the absolute value amplifier is exceeded. Therefore the threshold of the EMI detection (V_{R}) should be set somewhere below the range of the absolute value amplifier 40 minus threshold voltage (V_{T}). Once the EMI exceeds threshold voltage (V_{T}) the delay timer 42 starts and after T seconds the output goes active and the alarm sounds.

If the input to the timing circuit 42 stays high for greater than T seconds, as controlled by the delay circuit 46, alarm circuit 36 would be activated and continue for at least Y seconds as controlled by one-shot 48 in conjunction with OR gate 50. Provisions could be made in the event that the device remains influenced by low level EMI for a long period of time. For example, the CPU could disable the alarm after a period of time and again arm it after another period of time.

The input to comparator 52 is applied to the alarm circuit 36 and is used to control an intensity aspect of the alarm. For example, in the case of a continuing increase in detected EMI, the input to the positive terminal of comparator 52 increases. This increased signal level at the input to comparator 52 may be coupled to the alarm 36 and may be used to increase the volume of the alarm 36 as the magnitude of the detected EMI increases (e.g., as the user nears the source or sources of disruptive EMI). This allows the patient to take appropriate action to move in a different direction to avoid exacerbating the EMI situation. Likewise, with a stimulative alarm, such as a vibratory alarm, the amount of vibration may be increased in response to increased EMI.

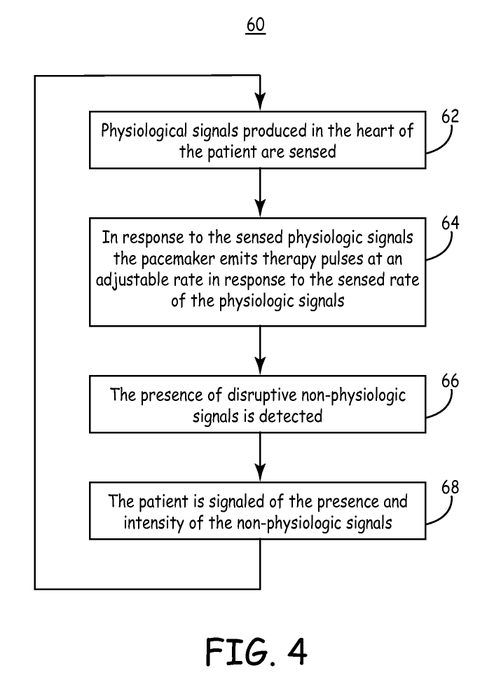

FIG. 4 is a flow chart 60 describing the method of operation of the present invention. In an implantable IMD coupled to the heart of a patient, the physiologic signals produced in the heart of the patient are sensed 62. In response to the sensed physiologic signals the pacemaker emits therapy pulses at an adjustable rate in response to the sensed rate of the physiologic signals 64. The presence of disruptive non-physiologic signals is detected 66, and the patient is signaled 68 of the presence of the non-physiologic signals so that the patient may move away from the source of such signals. The signaling may be of any of several forms as noted above, including, but not limited to tactile signaling and visual or audible signaling. Also as noted above, the present invention provides for an alarm the increases in intensity in response to an increase in the level of EMI detected.

The method herein may be performed as a processor-controlled method wherein a set of executable instructions stored on a computer-readable medium cause the desired outcome. That is, detection of deleterious EMI and signaling a patient when said EMI exceeds a predetermined threshold. For example, the instructions stored on the computer-readable medium may comprise the following instructions:

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. An implantable medical device, comprising:
a sensor responsive to physiologic conditions of a patient;
therapy delivery circuitry coupled to the sensor and providing therapy in response to the sensed physiologic conditions;
a detector coupled to the sensor and detecting electromagnetic interference and providing a signal indicating the present magnitude of the detected electromagnetic interference;
an alarm perceptible to the patient and coupled to the detector, activated responsive to the signal indicating that the present magnitude of the detected electromagnetic interference exceeds a defined threshold; and
control circuitry coupled to the alarm and the sensor ceasing activation of the alarm responsive to the signal indicating that the present magnitude of the detected electromagnetic interference is below the defined threshold.

2. A device according to claim 1 wherein the alarm is a stimulative alarm.

3. A device according to claim 1 wherein the alarm is an auditory alarm.

4. A device according to claim 1 wherein the sensor is responsive to cardiac signals.

5. A device according to claim 4 wherein the therapy circuitry comprises cardiac pacing circuitry.

6. A device according to claim 4 wherein the therapy circuitry comprises cardiac defibrillation circuitry.

7. A device according to claim 1 wherein the alarm increases in intensity responsive to the signal indicating an increase in the present magnitude of the detected electromagnetic interference.

8. A device according to claim 1 wherein the alarm is activated for at least a predefined time period responsive to the signal indicating that the present magnitude of the detected electromagnetic interference exceeds a defined threshold.

9. A method of increasing safety of a patient having an implantable medical device, wherein the device comprises:
a sensor responsive to physiologic conditions of a patient;
therapy delivery circuitry coupled to the sensor and providing therapy in response to the sensed physiologic conditions;
a detector coupled to the sensor and detecting electromagnetic interference and providing a signal indicating the present magnitude of the detected electromagnetic interference; and
a patient perceptible alarm, the method comprising:
activating the alarm perceptible to the patient responsive to the signal indicating that the present magnitude of the detected electromagnetic interference exceeds a defined threshold; and
ceasing activation of the alarm responsive to the signal indicating that the present magnitude of the detected electromagnetic interference is below the defined threshold.

10. A method according to claim 9 wherein the alarm is a stimulative alarm.

11. A method according to claim 9 wherein the alarm is an auditory alarm.

12. A method according to claim 9 wherein the sensor is responsive to cardiac signals.

13. A method according to claim 12 wherein the therapy circuitry comprises cardiac pacing circuitry.

14. A method according to claim 12 wherein the therapy circuitry comprises cardiac defibrillation circuitry.

15. A method according to claim 9 comprising the alarm increasing the intensity of the alarm responsive to the signal indicating an increase in the present magnitude of the detected electromagnetic interference.

16. A Method according to claim 9 comprising activating the alarm for at least a predefined time period responsive to the signal indicating that the present magnitude of the detected electromagnetic interference exceeds a defined threshold.
